(19) United States
(12) Patent Application Publication
(10) Pub. No.: US 2019/0122751 A1
(43) Pub. Date: Apr. 25, 2019

(54) DECISION ORGANIZER FOR PRIORITIZING RESOURCE ALLOCATION

(71) Applicant: The Quantum Group Inc., Lake Worth, FL (US)

(72) Inventors: Noel GUILLAMA, Wellington, FL (US); Chester HEATH, Boca Raton, FL (US)

(21) Appl. No.: 16/165,862

(22) Filed: Oct. 19, 2018

Related U.S. Application Data
(63) Continuation-in-part of application No. 15/826,304, filed on Nov. 29, 2017.
(60) Provisional application No. 62/574,523, filed on Oct. 19, 2017.
(60) Provisional application No. 62/427,500, filed on Nov. 29, 2016.

(51) Int. Cl. G16H 10/60; G06F 9/50

(52) U.S. Cl. CPC G16H 10/60; G06F 9/5011

(57) ABSTRACT

The present disclosure provides for systems and methods to prioritize allocations of resources between a plurality of vendors and a plurality of receivers. The vendors can provide products or services and can desire to contract with the receivers to coordinate economic transactions for all of the vendors' products or services. The receivers can be individuals or entities who seek to receive products or services from the vendors according to preferences of the specific receiver. For example, a receiver can prefer to select a desired product or service from the vendors according to cost, time of availability, location, and a host of other variables. In addition, the present disclosure can provide for a payment structure to integrate different payment methods of both the plurality of vendors and the plurality of receivers.

### CROSS REFERENCE TO RELATED APPLICATIONS

This application claims priority to and the benefit of U.S. Provisional Application No. 62/574,523, entitled, “Medical Decision Organizer: System for the Arbitrage of Medical Resources, Commodities, and Services,” filed Oct. 19, 2017 the contents of which are incorporated by reference in their entirety as if fully set forth herein. This application is a continuation-in-part of co-pending U.S. patent application Ser. No. 15/826,304, filed Nov. 29, 2017, entitled, “Decision Organizer”, which claims priority to and the benefit of U.S. Provisional Application No. 62/427,500, entitled, “Decision Organizer,” filed Nov. 29, 2016. Each of the foregoing applications is herein incorporated by reference in their entirety as if fully set forth herein.

### FIELD

The present disclosure is related to the field of computer-based processing, and more particularly, to prioritized allocation of resources.

### BACKGROUND

Society has greatly benefited from advances in medical technology, equipment, techniques and processes. Despite these advances, a significant challenge facing the medical field is a cost effective allocation of these resources. Fundamentally, advanced technology is expensive but patients require continuously available access to medical treatment, services, and products.

In particular, sophisticated diagnostic equipment is often financed to make it affordable. Financing can cause some medical equipment to cost thousands of dollars per day whether it is used productively or not. However, the usable lifetime of the equipment is modulated by ongoing advances in technology, which can quickly make the equipment obsolete. Consequently, there is a critical need to allocate high technology resources for maximum utilization by patients.

Today, medical care is not equally affordable by all prospective patients. For many patients, advanced medical technology can be out of reach; these patients can be unfairly limited in their access to affordable, appropriate healthcare services and treatment. Care providers are often in the middle of this cost-need conflict, with few tools to optimize the care providers' need to finance their equipment and to provide access to the technology for less affluent patients. Generally, the more patients receiving access to the technology, the higher the utilization of the equipment and the higher the productivity of the financing. This is true even some portion of the patients cannot fully compensate for the full, pro-rated cost of the treatment that they receive. It is financially beneficial for health care providers to keep the equipment and recoup any amount of payment at all.

For example, assume an expensive diagnostic tool is potentially available in continuous cycles 24×7, and operating technicians can be provided with minimal expense, even at odd hours. Further, this tool requires several cycles to initialize at a significant cost; however, the same tool can be ready for the next cycle of use, with minimal latency, at much lower cost. Therefore, continuous operation is desirable. An additional patient, inserted at the end of a continuous demand period, can enjoy the savings afforded by avoiding an unnecessary setup delay and expense. For example, productivity of the tool is increased when a gap in scheduled use is filled at all, even by a lower paying patient. However, if the machine must be shut down due to lack of scheduled use, the next appointment will have a high initialization cost. Therefore, it is desired that all of the open appointments be concentrated in that period.

However, care providers do not currently have systems or methods which allow for locating patients, and determining how to attract lower-paying patients to fill gaps in their schedules. Additionally, patients have no ability to compare schedules of care providers in order to find a care provider who can provide them with a lowest rate for a service.

### SUMMARY

The present invention is directed to systems and methods for fairly arbitrating need versus availability decisions with the objective of optimal utilization of medical resources and delivery to the greatest number of qualified patients. A first embodiment of the invention can provide for a Decision Organizer that integrates a complex tradeoff or arbitrage of the requirements and flexibility of a plurality of patients against the costs of service delivery and incentives for a plurality of service providers or vendors.

A second embodiment of the present disclosure can provide a system that performs a similar function for a plurality of specialized care givers, with specific skills and availability, against the requirements of a plurality of organizations, representing patients, requiring those specializations. The second embodiment can provide for decision-making based on technical expertise and risk management.

A third embodiment of the present disclosure can be a complex triage decision made with a major disaster or act of war. The decisions made by an exemplary decision organizer can include selecting and allocating qualified institution and specific skills. The decision organizer can weigh any risks involved with each option against the probability of success for each option and determine an optimal path.

In some examples of the third embodiment, economics can be a null factor.

In some examples of the third embodiment, a mechanism for reservation and allocation of resources can exist which is funded by government.

In other examples of the third embodiment, the decision organizer can provide for assignment of scarce durable medical equipment is open to arbitrage as well.

A fourth embodiment of the present disclosure can provide for organizing the combined availability of multiple health care entities. The entities can include diagnostic systems, specific medical skills, specialized care givers, supplies, hospitals, qualified medical centers, recovery facilities, and equipment. Each of these entities can be used to complete a patient's care from diagnosis to recovery and then through follow-up education.

A fifth embodiment of the present disclosure can include a system. The system can include an arbitrage manager, a plurality of user terminals, and a plurality of service systems. The plurality of user terminals can be associated with a plurality of patients and communicatively coupled to the arbitrage system. The plurality of service systems can be associated with a plurality of medical service providers and can be communicatively coupled to the arbitrage system. The arbitrage system can be configured to complete a series of steps. The steps can include first obtaining, via the user terminals, patient information for each of the plurality of patients. The patient information can indicate requirements for medical services and flexibility regarding the requirements. The steps can then provide for collecting, from the plurality of service systems, service information for the plurality of medical service providers, the service information comprising available services, costs and availability for the available services, and incentives available. The system can then provide for generating one or more proposals for the medical services for each of the at least one patient. The proposals can be based on the patient information and the service information. The proposals can be generated so as to maximize a utilization of the each of the plurality of medical service providers.

A sixth embodiment of the present disclosure can provide for a method which can include a series of steps. The steps can provide for first obtaining, via a plurality of user terminals associated with a plurality of patients, patient information for each of the plurality of patients. The patient information can indicate requirements for medical services and flexibility regarding the requirements. The steps can then provide for collecting, from a plurality of service systems associated with a plurality of medical service providers, service information for the plurality of medical service providers. The service information can include available services, costs and availability for the available services, and incentives available. The steps can then provide for generating one or more proposals for the medical services for each of the at least one patient. The proposals can be based on the patient information and the service information. The proposals can be generated so as to maximize a utilization of the each of the plurality of medical service providers.

### BRIEF DESCRIPTION

The accompanying drawings exemplify the embodiments of the present invention and, together with the description, serve to explain and illustrate principles of the invention. The drawings are intended to illustrate major features of the exemplary embodiments in a diagrammatic manner. The drawings are not intended to depict every feature of actual embodiments nor relative dimensions of the depicted elements, and are not drawn to scale.

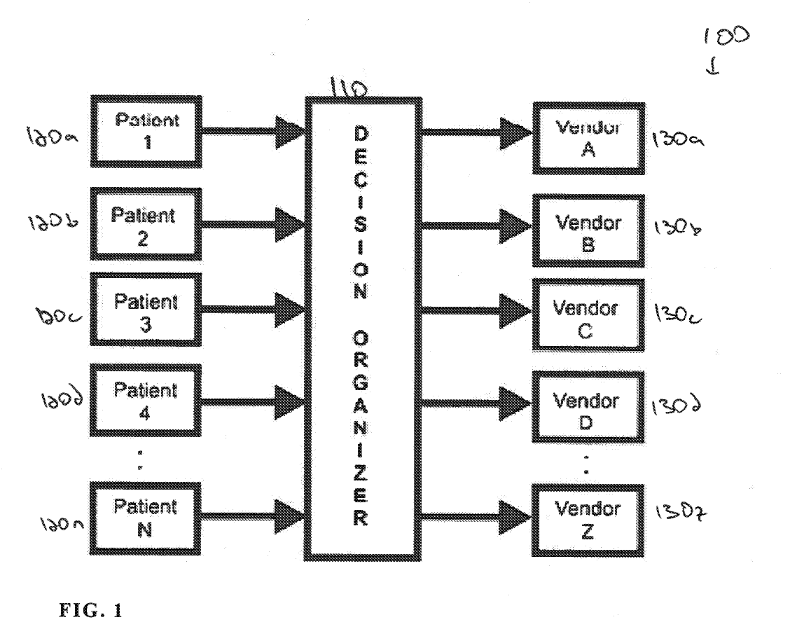

FIG. 1 shows a schematic overview of an exemplary decision organizer, according to an embodiment of the present disclosure,

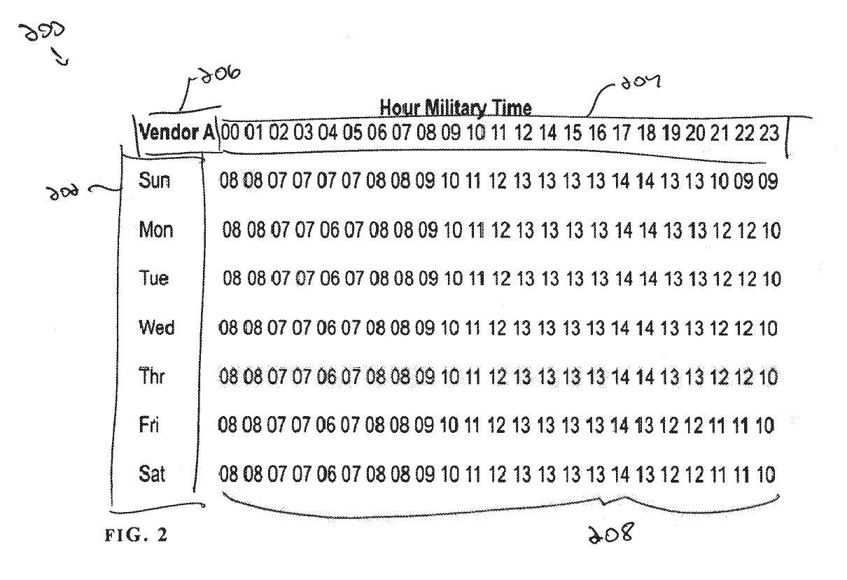

FIG. 2 shows an exemplary rate chart, according to an embodiment of the present disclosure.

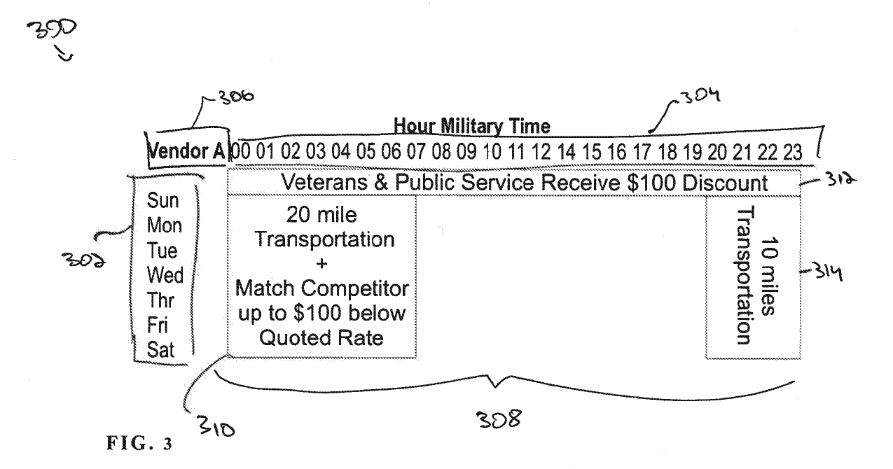

FIG. 3 shows an exemplary rate chart with conditional incentives, according to an embodiment of the present disclosure.

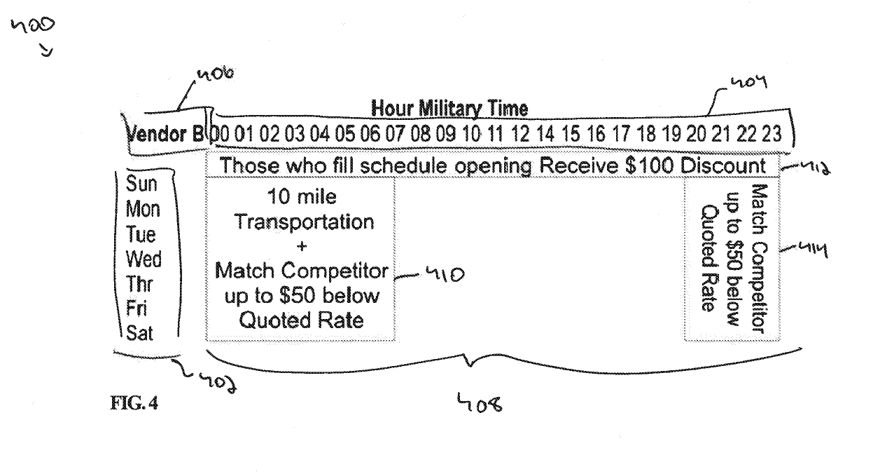

FIG. 4 shows an exemplary rate chart with conditional incentives, according to an embodiment of the present disclosure.

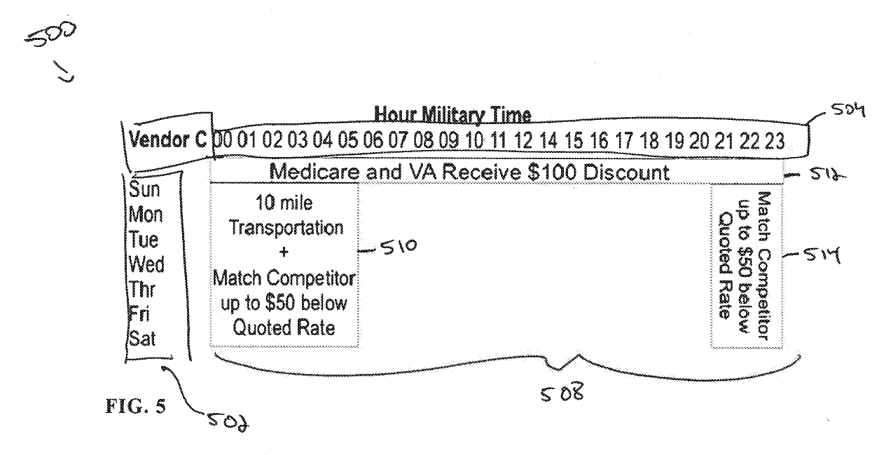

FIG. 5 shows an exemplary rate chart with conditional incentives, according to an embodiment of the present disclosure.

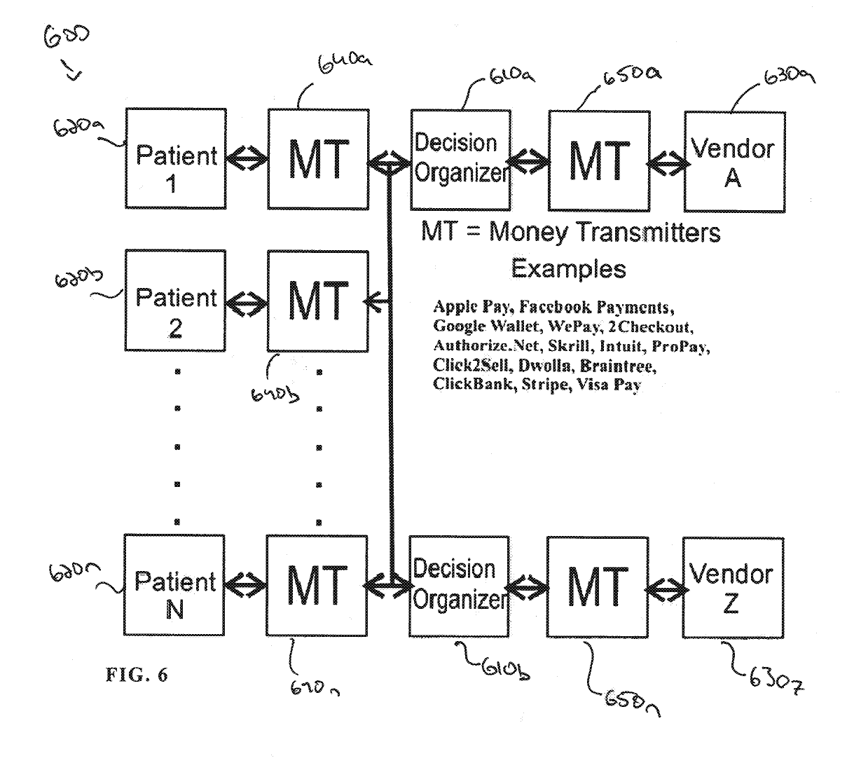

FIG. 6 shows an exemplary payment structure, according to an embodiment of the present disclosure.

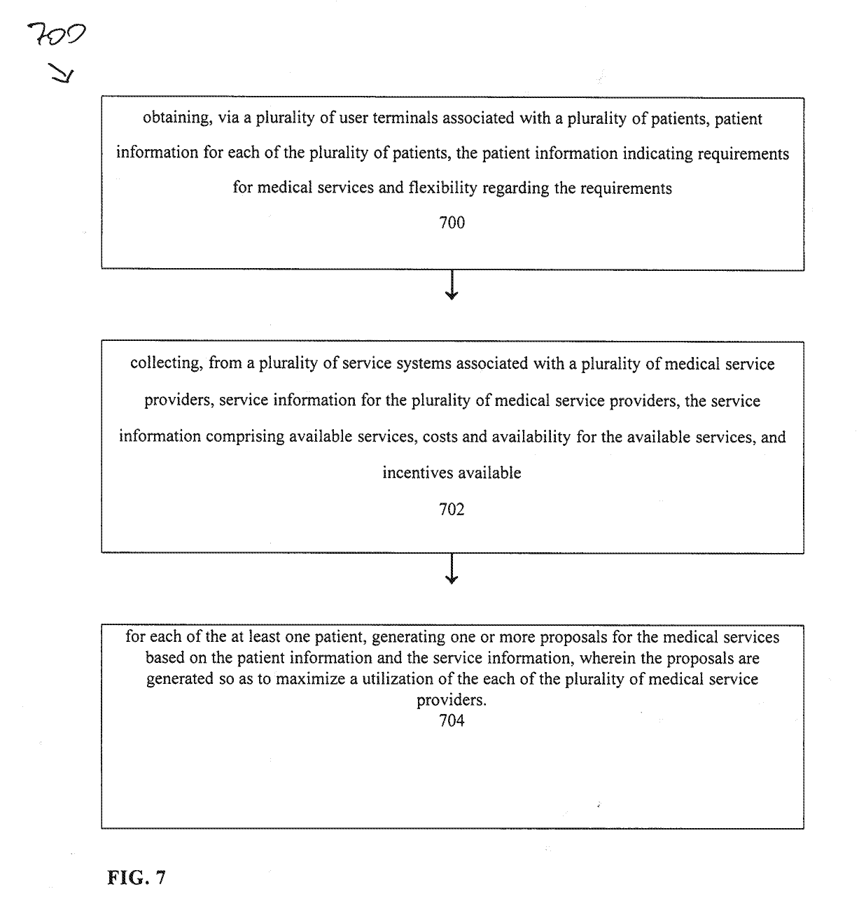

FIG. 7 is a flowchart of steps in a method according to the various embodiments.

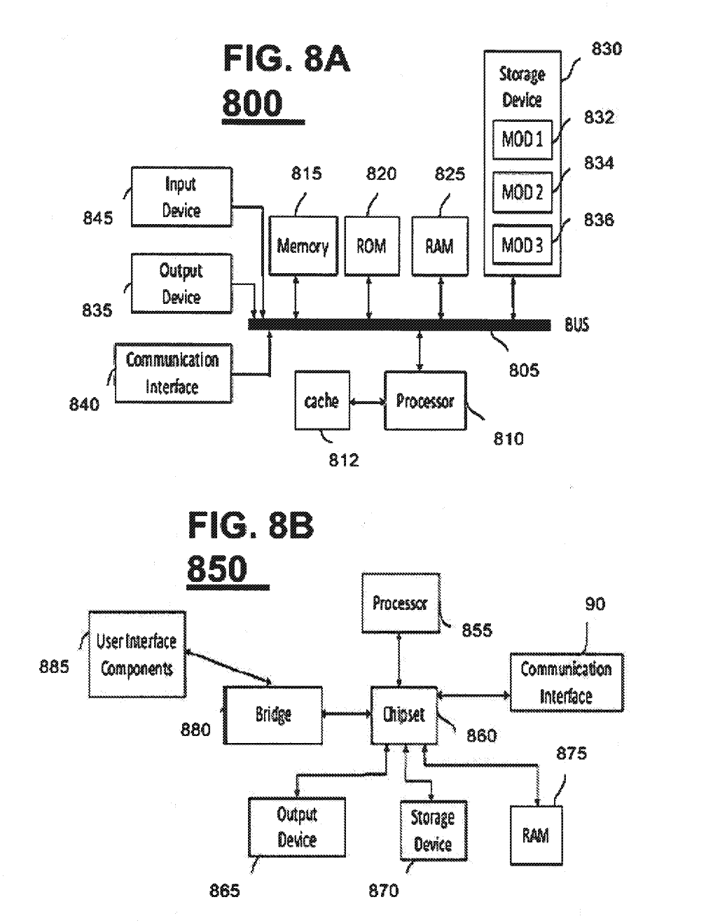

FIG. 8A illustrates a conventional system bus computing system architecture.

FIG. 8B illustrates a computer system having a chipset architecture.

### DETAILED DESCRIPTION

The present invention is described with reference to the attached figures, where like reference numerals are used throughout the figures to designate similar or equivalent elements. The figures are not drawn to scale, and are provided merely to illustrate the instant invention. Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details, or with other methods. In other instances, well-known structures or operations are not shown in detail to avoid obscuring the invention. The present invention is not limited by the illustrated ordering of acts or events, as some acts may occur in different orders and/or concurrently with other acts or events. Furthermore, not all illustrated acts or events are required to implement a methodology in accordance with the present invention.

The present disclosure provides for systems and methods to prioritize allocations of resources between a plurality of vendors and a plurality of receivers. The vendors can provide products or services and can desire to contract with the receivers to coordinate economic transactions for all of the vendors' products or services. The receivers can be individuals or entities who seek to receive products or services from the vendors according to preferences of the specific receiver. For example, a receiver can prefer to select a desired product or service from the vendors according to cost, time of availability, location, and a host of other variables. In addition, the present disclosure can provide for a payment structure to integrate different payment methods of both the plurality of vendors and the plurality of receivers.

FIG. 1 shows a schematic overview of an exemplary decision organizer system 100, according to an embodiment of the present disclosure. System 100 can include a decision organizer 110; a plurality of receivers, 120a, 120b, 120c, 120d, . . . and 120n; and a plurality of vendors 130a, 130b, 130c, 130d, . . . and 130z.

System 100 provides a simplified view of the interactions between receivers 120a, 120b, 120c, 120d, . . . and 120n and a plurality of vendors 130a, 130b, 130c, 130d, . . . and 130z. Any number of receivers 120a, 120b, 120c, 120d, . . . and 120n and vendors 130a, 130b, 130c, 130d, . . . and 130z can integrate with the system 100.

Each receiver 120a, 120b, 120c, 120d, . . . and 120n can seek to engage with the market for a desired product or service and have a set of preferences about the product or service. Each receiver 120a, 120b, 120c, 120d, . . . and 120n can be an individual, a group of individuals, a business enterprise, or any organization wishing to engage in the market. For example, the preferences can include needs, qualifications, requirements, and flexibility. Each vendor 130a, 130b, 130c, 130d, . . . and 130z can be any individual, group of individuals, business, or organization on the market to sell, license, or vend a plurality of products or services according to a set of preferences. The preferences can include conditions and requirements for the products and services.

The decision organizer 100 can take into account all of the preferences of the plurality of receivers 120a, 120b, 120c, 120d, . . . and 120n and all of the preferences of the plurality of vendors 130a, 130b, 130c, 130d, . . . and 130z to determine an optimal match such that all receivers 120a, 120b, 120c, 120d, . . . and 120n can receive their desired product or service and vendors 130a, 130b, 130c, 130d, . . . and 130z get the opportunity to vend their products or services.

In some examples of the present disclosure, the receivers 120a, 120b, 120c, 120d, . . . and 120n can be patients and the vendors 130a, 130b, 130c, 130d, . . . and 130z can be care providers.

Patients can make use of the decision organizer 110, because the decision organizer 110 can evaluate every possible option on the market and automatically suggest a best fit for the individual patient, based on the patient's preferences. Therefore, the decision organizer 110 can work as a patient's advocate across a range of vendors 130a, 130b, 130c, 130d, . . . and 130z in the market. In some examples of the present disclosure, the decision organizer 110 can retrieve discounts for particular receivers 120a, 120b, 120c, 120d, . . . and 120n from particular vendors 130a, 130b, 130c, 130d, . . . and 130z. The decision organizer 110 can determine, for example, the lowest possible prices of a service or product. The decision organizer 110 can provide an exhaustive, global view of the selection process for a receiver 120a, 120b, 120c, 120d, . . . or 120n. Altogether, the decision organizer offers advice to receivers 120a, 120b, 120c, 120d, . . . and 120n while allowing each individual receiver 120a, 120b, 120c, 120d, . . . and 120n to make the final selection.

In some examples of the present disclosure, the decision organizer 110 can integrate with the internet or a loaded database of available receivers 120a, 120b, 120c, 120d, . . . and 120n and vendors 130a, 130b, 130c, 130d, . . . and 130z. A receiver 120a, 120b, 120c, 120d, . . . and 120n can access the decision organizer 110 from any PC, handheld device, smart device, tablet, or any means of accessing the internet.

Vendors 130a, 130b, 130c, 130d, . . . and 130z can use the decision organizer 110 to bring the vendors 130a, 130b, 130c, 130d, . . . and 130z business that from receivers 120a, 120b, 120c, 120d, . . . and 120n that the vendors they might not otherwise 130a, 130b, 130c, 130d, . . . and 130z have been successful at accessing. For example, receivers 120a, 120b, 120c, 120d, . . . and 120n are not diligent about exhaustively searching for the perfect vendor, and typically just evaluate a couple options before making a selection. Therefore, vendors 130a, 130b, 130c, 130d, . . . and 130z are often unable to access receivers 120a, 120b, 120c, 120d, . . . and 120n who might actually be interested in a vendors' 130a, 130b, 130c, 130d, . . . and 130z product.

In some examples of the present disclosure, vendors 130a, 130b, 130c, 130d, . . . and 130z can target particular receivers 120a, 120b, 120c, 120d, . . . and 120n with customized promotions and sales. For example, a decision organizer 110 can alert a vendor 130a, 130b, 130c, 130d, . . . and 130z that a receiver 120a, 120b, 120c, 120d, . . . and 120n entered the market for a particular product or service. Vendors 130a, 130b, 130c, 130d, . . . and 130z can then provide an offer or incentive directly to the interested receiver 120a, 120b, 120c, 120d, . . . and 120n only. In some cases, the decision organizer 110 can alert vendors 130a, 130b, 130c, 130d, . . . and 130z of the receivers' 120a, 120b, 120c, 120d, . . . and 120n preferences.

An exemplary decision organizer 110, according to an embodiment of the present disclosure can have no inventory and operate solely as an intermediary in the marketplace. Decision organizer 110 can scale effortlessly to increased numbers of receivers 120a, 120b, 120c, 120d, . . . and 120n and vendors130a, 130b, 130c, 130d, . . . and 130z.

Therefore, system 100 can provide a means for receivers and vendors to contact each other and conduct personalized transactions according to the preferences and availabilities of each party. Such a system can manage an exhaustive list of complicated variables and preferences for both receivers and vendors. This exhaustive list involves many more variables than the human mind can accurately balance and can provide an optimal solution where individuals do not bother to find an actual optimal solution. System 100 can further store the preferences or needs of receivers. For example, system 100 can include a machine parse-able database of medical profile and history.

FIG. 2 shows an exemplary rate chart 200 for a vendor A, according to an embodiment of the present disclosure. Rate chart 200 can include a listing of available days 202; a plurality of time slots 204; a vendor identifier 206; and a plurality of rates 208.

The vendor identifier 206 can associate the chart 200 with a particular vendor who is willing to conduct transactions according to the prices listed in the chart 200. A plurality of rates 208 can be included in the chart 200 in slots corresponding to appropriate days 202 and time slots 204. The plurality of rates 208 can be estimated based on known, or predictable, patterns of demand. The plurality of rates 208 can vary with the days 202 and time slots 204.

FIGS. 3-5 show how exemplary rate charts can be modified by vendors seeking to attract particular receivers. FIG. 3, for example, shows a chart 300 which includes a listing of available days 302; a plurality of time slots 304; a vendor identifier 308; a plurality of rates 308; and a plurality of incentives 310, 312, and 314. Components of FIG. 3 can be as provided for by similarly named elements of FIG. 2. In addition, chart 300 can include a transportation and competitor-matching incentive 310; a personnel discount 312; and a transportation incentive 314. FIGS. 4-5 can include many components and similar labels to chart 300 of FIG. 3 and can be provided for according to the corresponding labels. FIG. 4 is an example of an alternate (B) vendor's conditional incentive chart 400 and FIG. 5 is an example of an additional alternate (C) vendor's conditional incentive chart 500.

For example, when a receiver can easily fit into an unfilled time slot 204, additional incentives 310, 312, and 314 can be added. This process of matching receivers to time slots and providing individualized incentives can provide some patients productivity advantages when compared to other patients who cannot aid the efficiency of utilization of the technology resource.

Furthermore, healthcare skills and techniques have similar concerns with the cost and latency of initialization, where similar improvements in productivity can be applied. The availability durable medical equipment and of entire medical centers can benefit from such a process as provided for with respect to FIGS. 1-5.

An example operation of the present disclosure is provided for below, with respect to FIGS. 1-5. The example operation shows advantages and benefits of the present disclosure for both vendors and receivers.

For example, an exemplary Patient #1 can seek services, according to an embodiment of the present disclosure. Patient #1 can interface with the decision organizer 110 to meet his needs. Patient #1 can have a plurality of preferences or characteristics, including (1) he requires transportation within 10 miles, (2) he is a fireman, (3) he needs a non-time-intensive test, (4) he is flexible enough to come into a (off a predictable) shift within a 12-hour notice to fill an opening, and (5) he will tolerate being moved within certain non- work days.

An exemplary Patient #2 can seek services at a similar time to Patient #1's request, according to an embodiment of the present disclosure. Patient #2 can also have a plurality of preferences or characteristics, including (1) he must have the test before potentially urgent surgery (a priority situation) scheduled as soon as the next day, after 4 pm (2) he has very little flexibility, (3) he needs no transportation, and (4) he can potentially cover a cancellation at any time during the day (until 6pm) if there is an advantage. Vendor A (Tri-County MRI), as provided for in chart 200 of FIG. 2 and chart 300 of FIG. 3 can be an exemplary dominant player in the area of patients #1 and #2.

Patients #1 and #2 can seek rates or offers from the vendors through the decision organizer 110. For example, perhaps all other competing vendors initially quoted rates are similar to Vendor A's rates in chart 200 of FIG. 2. However, perhaps Vendor B's default rate chart is $100 less than Vendor A on weekdays. Vendor B (St. Mary's MRI) offers technicians a weekend shift bonus and charges the same as Vendor A on weekends to pay for it. Vendor C charges $100 more at every hour than Vendor A, but charges $50 less than Vendor A's default rate on weekdays to pay for the weekend shift bonus. Vendor D simply matches everything that Vendor A offers.

The rates vary between vendors, and competitive incentives can potentially be offered, according to another policy chart for vendors (such as charts 300, 400, and 500, as provided for in FIGS. 3-5). Chart 300 shows that Vendor A offers taxi transportation (10 or 20 miles one way) during evening hours and that Vendor A will match any other offer along with a $100 discount to service personnel. Vendor A can further offer other benefits not shown in FIG. 3, including (1) if the patient is delivered to the facility, and transportation is not required, there is a rebate of $50, (2) Vendor A will conditionally match a competitor's quote up to $100 below Vendor A's quote except during peak hours (8 am to 5 pm), and (3) Vendor A can require $100 to hold a time slot reservation, and (4) Vendor A will refund $100 to move an appointment in Triage situation.

Vendor B is “hungrier” and offers a different set of potential incentives in chart 400 of FIG. 4. Vendor B's rate table is more aggressive than Vendor A and Vendor B can match any competitor during off peak hours. If an opening is filled (within 12 hours notice), Vendor B offers another $100 discount and $200 if a scheduling hole is filled.

Vendor C's preferences are shown in chart 500 of FIG. 5. Vendor C offers similar incentives as vendor B, but incents Medicare and VA private patients $100. Vendor C also defines “peak” hours differently (6 am to 8 pm). Vendor D (Central Hospital) mimics Vendor A basic rate across the board, has the best location for in-house patients yet offers no incentives.

Vendors A, B, and C can charge an additional $100 fee to make or break reservation within 12 hours' notice. Vendors sometimes can manually observe competition and present immediate incentives to move an appointment, in order to provide the longest period of continuity for cost effectiveness.

However, these promotions are not initially offered, they are conditional on competitor's offers. In order to assure a reasonable profit for the services, the promotions will appear in response to competition, or perhaps spontaneously if enough time has passed and no closure occurs, or in response to a question posed by a patient.

The rates and incentives reflect the efficiencies that vendors estimate. All vendors can see a patient's profile, time requirements, and what other vendor competition is offering, so all vendors are forced to be competitive. The customer experience is therefore interactive because promotions can occur in response to a vendor's ability to complete a schedule. Other conditions, such as ability to pay, cash payment, income, credit history, or even friends and family conditions can be included. Further there can be spot promotions for using the decision organizer 110 service or referral from specific providers.

There could be additional rules imposed by vendors. Exemplary rules can include starting a bid at $X100 and reducing automatically by 5% each automated bid, with a maximum discount limit. If customer will do multiple times or ranges (example midnight to 3 AM Monday-Friday) then a vendor can choose to provide a maximum percentage discount. Discounts can be provided for any time not past midnight or more than N miles away, where N is a variable distance chosen by the vendor. Discounts can be provided for filling an opening immediately within hour, or filling an opening tomorrow.

Such a list of rules and preferences for both the vendors and the receivers can increase exponentially as more vendors and receivers integrate with system 100 and as each party develops additional rules and confirms more appointments. A human is incapable of handling this complexity and does not go through such an exhaustive chart to make a selection. Therefore, the present disclosure provides advantages over conventional, human methods.

Altogether, a customer will experience an interactive experience as vendors compete to fill their schedules. Patients can even receive spot advertising such as: “Convenient-adjacent to St. Mary's Hospital”. Or warnings: “This Vendor C's testing facility will not accommodate anyone over the weight of 190 lbs or 6 feet tall”. The process can also offer advice: “This equipment is preferable for the particular testing you require”. In some examples, once a vendor agrees to an appointment, vendors cannot withdraw from the agreed appointment. However, inventors can incent a patient to change.

Another user experience with system 100 of FIG. 1 can be provided for as below. Patient #1 is 6 foot 4″ tall and 210 lb, 29 year old firemen, needing a non-urgent workplace certification test. His shift begins at 6pm Friday and he needs to schedule for 4 pm. He is initially quoted: 1300 less $100 Public Service Discount=$1200 (selected) for Vendor A; 1200 (matching A) for Vendor B; no bid—based on profile (too big) for Vendor C; and $1300 for Vendor D. He can select Vendor B at 4 pm.

Then Patient #2, a VA patient urgently needs the 4 pm slot at the last minute and is quoted: $1400 for Vendor A; $1200 (1200−100 (Medicare)+100 (last minute) for Vendor B; $1450 for Vendor C; and $1400 for Vendor D. Patient #2 can then also select Vendor B at 4 pm.

However, this bumps Patient 1 to 5 pm. Patient 1 can no longer make his 6 PM work shift and must move to 4 PM Tuesday after his shift ends as all other alternate 4 pm slots are filled. Patient 1 is then quoted: $1300 less $100 Public Service Discount=$1200−less $100 to move appointment=1100 for Vendor A; $1200 (matching A) for Vendor B; No Bid−based on profile (too big) for Vendor C; and $1300 on original schedule for Vendor D.

At the same time as Patient 1 attempting to reschedule, Patient 2 is offered an incentive to move to an open 5 pm slot. Patient 2, a VA patient can accept the 5 pm slot at the last minute and is quoted: $1100 because the last minute penalty no longer applies—less move incentive for Vendor A; $1200 (1200-100 (Medicare)+100 (last minute) @4 pm for Vendor B; $1450 @4 pm for Vendor C; and $1100 @4 pm for Vendor D.

Either vendor A or D offers the lowest price for patient 2; patient 2 can select the better location and time of Vendor D. Patient 2 saves $100 and his results can still be available for his next day procedure. The lowest rate is $800 less $100 (best incentive) and this reflects the threshold of profit because both vendors profit.

Such a determination, as provided for above, can have far greater complexity when scheduling appointments for 24×7 time slots for a month (722 appointments average) for as many patients. Only a computer could handle this complex determination.

A computer system, as provided for with respect to the present disclosure can both arbitrate a very complex set of rules, and can also interact with human overrides. For example, the urgent (VA) patient needs further investigation and there is a choice between a cardiologist and a cardiac surgeon. A similar arbitration of rates to the above can occur, but further the payer can intercede if the individual risk histories of each specialist exceed a limit (such that complications are more likely to occur)

The present disclosure can further provide for complex trade-off situations, according to an embodiment of the present disclosure. For example, arbitration for fees can work as before to yield a combination of cardiologist and cardiac surgeon at lowest cost. However, either a sum of the risk histories or a computer simulation of interaction predicts an excessive complication rate (which can actually be most expensive to the payer overall) is rejected by the payer's rule set. This would be the same situation if two specialists refuse to work with each other. Further, the results of one specialist can require only one alternative of another. Alterntaively, there can be a human override where a second specialist either chooses or refuses to accept the situation.

For example, the cardiologist determines a severe blockage of 5 coronary arteries that only one local surgeon has the skills to perform or will accept the assignment—or given the challenge, one surgeon volunteers to accept the more difficult procedure. For example, the most qualified cardiac surgeon to perform the VA patient's surgery has available time, but is not nearby. Remote Control Robotic surgery provides a potential solution. The preferred surgeon is at University Hospital and the critical VA patient is at Central Hospital and only that Doctor has the credentials (or is estimated to have the lowest complication rate and overall lowest cost of the total process by simulation against other past experiences) to operate the equipment.

The system 100 can be alerted that the cost of the procedure exceeds a limit that the payer will accept at standard quoted rates. The system 100 can then seek volunteers to quote a lower rate or do the surgery pro bono. These are all examples of complex tradeoff decisions where automated arbitration by a decision organizer, according to an embodiment of the present disclosure, can provide a top down complex arbitrage of alternatives. Further, the decision organizer makes the best and most cost-effective use of advanced skills and equipment for the community at large. An exemplary decision organizer can distribute resources most efficiently and productively. The decision maker can then manage complex triage situations and national emergencies, where humans could not accurately or efficiently evaluate the options and solutions

For example, there exist a finite number of cardiac surgeons who can provide bypass operations. Some of these surgeons can only do the operation “On Pump” only—meaning that they require the availability of a heart lung machine and operator. Other surgeons can perform the operation “Off Pump”, meaning that the operation is performed on a beating heart and a heart-lung machine is not required. The latter surgeon is more highly skilled and requires less support equipment. His fee can be higher. Also, the use of the heart-lung machine adds risk to the operation. Therefore, in some examples of the present disclosure, surgeons can be considered vendors of the service.

In another example of the present disclosure, a hospital can have a panel of cardiologists, each representing patients with different needs and requirements. The cardiologists represent the patients on the left side of the decision organizer drawing.

FIG. 6 shows an exemplary payment system 600, according to an embodiment of the present disclosure. System 600 can include decision organizers 610a and 610b; a plurality of receivers 620a, 620b, . . . and 620n; a plurality of money transmitters corresponding to the receivers, 640a, 640b, . . . and 640n; a plurality of money transmitters corresponding to the vendors 650a and 650n; and a plurality of vendors 630a and 630b.

For minimal disruption to the current banking and electronic commerce means, the decision organizer 610 can function as a Buyer through which ever money transmitter 650a or 650n has an agreement with a given vendor 630a and 630b—and—also functions as a seller through one (or more) money transmitters 640a, 640b, . . . and 640n to the eventual buyer 620a, 620b, . . . and 620n. Decision Organizer 610 is therefore a transaction intermediary between one or more seller and one or more buyers 620a, 620b, . . . and 620n. It is a common language (Esperanto) effectively between sellers 630a and 630b and buyers 620a, 620b, . . . and 620n who may be restricted to a single protocol. For example, sellers 630a and 630b on eBay may be limited to Paypal transactions only, but buyers 620a, 620b, . . . and 620n, using only Google Wallet can purchase directly from vendors 630a and 630b for one or more purchases.

Therefore, system 600 provides a means for commerce, where commerce can be otherwise blocked by limited protocols. System 600 can act as an electronic purchase credit exchange, much like the Federal Reserve that allows Decision Organizer 610 to become a common shopping cart for the entire Internet.

A plan for commitment of these resources must be defined initially to assure their availability at the required times. These commitments can require advance payments for materials, reservation or initialization, or in reverse as compensation when adjustments for productivity, or scheduling, afford better efficiencies. Because many of the elements 620a, 620b, . . . 620n, 630a, and 630b can be separate businesses, there must be a means for payment through multiple web interfaces representing disparate entities with differing payment protocols. FIG. 6 defines an intermediary relationship through monetary transfer services that allows payment or reservation.

Acting as an intermediary, the Decision Organizer 610 can also work through the patient web portal of a medical information system and simultaneously through the website of the vendor as well. For minimal disruption to the current banking and electronic commerce means, Decision Organizer functions as a Buyer through which every money transmitter has an agreement with a given vendor and also functions as a seller through one (or more) money transmitters to the eventual buyer. Decision Organizer is therefore a transaction intermediary between one or more patients and one or more vendors. It is a common language (Esperanto) effectively between sellers and buyers who can be restricted to a single protocol. For example, sellers on eBay can be limited to Paypal transactions only, but buyers, using only Google Wallet can purchase directly from them for one or more purchases. Altogether, system 600 provides a means for universal commerce, where commerce is otherwise blocked by limited protocols.

Either end of the transaction can provide credit offers or the medical information system can provide billing.

System 600 can provide interactive negotiation between the patient and the medial testing service vendor with various algorithms and the ability to work through existing web portals and still enable billing for reservation or payment. System 600 provides the ability to do a top down optimal utilization of skills and equipment in situations that are beyond human conception, or responsiveness.

FIG. 7 is a flowchart of steps in a method according to the various embodiments. The method begins at step 700 in which an arbitrage system obtains, via a plurality of user terminals associated with a plurality of patients, patient information for each of the plurality of patients, the patient information indicating requirements for medical services and flexibility regarding the requirements. The method then goes to step 702, where the arbitrage system collects, from a plurality of service systems associated with a plurality of medical service providers, service information for the plurality of medical service providers, the service information comprising available services, costs and availability for the available services, and incentives available. Thereafter, at step 704, the arbitrage system generates, for each of the patients, one or more proposals for the medical services based on the patient information and the service information, wherein the proposals are generated so as to maximize a utilization of the each of the plurality of medical service providers.

Various embodiments of method 700 can include the ability to include real-time or stored-time utilization of computer simulation against risk or other factors in the complex decision and the ability to include alerts and human override to the system.

Within a site, a common purchase protocol is typically maintained, while the purchase protocol can vary radically between sites. To resolve this concern, once a protocol is exercised manfully, it is learned by Decision Organizer and saved for later use by other buyers who visit the same site on Decision Organizer. If the protocol fails, it reverts to manual operation and the new protocol is learned. Users can elect to do all transactions manually, allow the machine to bring them to an authorization screen, or have the system utilized different payment means for different sales. There is a finite number of common protocols used by many sites. Each of these protocols can be assigned a nickname for easy lookup. Sites can also adjust to Decision Organizer using a choice of predefined protocols offered.

FIG. 8A, and FIG. 8B illustrate exemplary possible system configurations. The more appropriate configuration will be apparent to those of ordinary skill in the art when practicing the present technology. Persons of ordinary skill in the art will also readily appreciate that other system configurations are possible.

FIG. 8A illustrates a conventional system bus computing system architecture 800 wherein the components of the system are in electrical communication with each other using a bus 805. Exemplary system 800 includes a processing unit (CPU or processor) 810 and a system bus 805 that couples various system components including the system memory 815, such as read only memory (ROM) 820 and random access memory (RAM) 825, to the processor 810. The system 800 can include a cache of high-speed memory connected directly with, in close proximity to, or integrated as part of the processor 810. The system 800 can copy data from the memory 815 and/or the storage device 830 to the cache 812 for quick access by the processor 810. In this way, the cache can provide a performance boost that avoids processor 810 delays while waiting for data. These and other modules can control or be configured to control the processor 810 to perform various actions. Other system memory 815 can be available for use as well. The memory 815 can include multiple different types of memory with different performance characteristics. The processor 810 can include any general purpose processor and a hardware module or software module, such as module 1 832, module 2 834, and module 3 836 stored in storage device 830, configured to control the processor 810 as well as a special-purpose processor where software instructions are incorporated into the actual processor design. The processor 810 can essentially be a completely self-contained computing system, containing multiple cores or processors, a bus, memory controller, cache, etc. A multi-core processor can be symmetric or asymmetric.

To enable user interaction with the computing device 800, an input device 845 can represent any number of input mechanisms, such as a microphone for speech, a touch-sensitive screen for gesture or graphical input, keyboard, mouse, motion input, speech and so forth. An output device 835 can also be one or more of a number of output mechanisms known to those of skill in the art. In some instances, multimodal systems can enable a user to provide multiple types of input to communicate with the computing device 800. The communications interface 840 can generally govern and manage the user input and system output. There is no restriction on operating on any particular hardware arrangement and therefore the basic features here can easily be substituted for improved hardware or firmware arrangements as they are developed.

Storage device 830 is a non-volatile memory and can be a hard disk or other types of computer readable media which can store data that are accessible by a computer, such as magnetic cassettes, flash memory cards, solid state memory devices, digital versatile disks, cartridges, random access memories (RAMs) 825, read only memory (ROM) 820, and hybrids thereof

The storage device 830 can include software modules 832, 834, 836 for controlling the processor 810. Other hardware or software modules are contemplated. The storage device 830 can be connected to the system bus 805. In one aspect, a hardware module that performs a particular function can include the software component stored in a computer-readable medium in connection with the necessary hardware components, such as the processor 810, bus 805, display 835, and so forth, to carry out the function.

FIG. 8B illustrates a computer system 850 having a chipset architecture that can be used in executing the described method and generating and displaying a graphical user interface (GUI). Computer system 850 is an example of computer hardware, software, and firmware that can be used to implement the disclosed technology. System 850 can include a processor 855, representative of any number of physically and/or logically distinct resources capable of executing software, firmware, and hardware configured to perform identified computations. Processor 855 can communicate with a chipset 860 that can control input to and output from processor 855. In this example, chipset 860 outputs information to output 865, such as a display, and can read and write information to storage device 870, which can include magnetic media, and solid state media, for example. Chipset 860 can also read data from and write data to RAM 875. A bridge 880 for interfacing with a variety of user interface components 885 can be provided for interfacing with chipset 860. Such user interface components 885 can include a keyboard, a microphone, touch detection and processing circuitry, a pointing device, such as a mouse, and so on. In general, inputs to system 850 can come from any of a variety of sources, machine generated and/or human generated.

Chipset 860 can also interface with one or more communication interfaces 890 that can have different physical interfaces. Such communication interfaces can include interfaces for wired and wireless local area networks, for broadband wireless networks, as well as personal area networks. Some applications of the methods for generating, displaying, and using the GUI disclosed herein can include receiving ordered datasets over the physical interface or be generated by the machine itself by processor 855 analyzing data stored in storage 870 or 875. Further, the machine can receive inputs from a user via user interface components 885 and execute appropriate functions, such as browsing functions by interpreting these inputs using processor 855.

It can be appreciated that exemplary systems 800 and 850 can have more than one processor 810 or be part of a group or cluster of computing devices networked together to provide greater processing capability.

### EXAMPLES

A person skilled in the art can readily understand that a decision organizer as provided for with respect to the present disclosure can be used across a variety of fields and industries.

For example, a decision organizer in the finance industry can allow the user to trade and compare with multiple online brokers, commodities, multiple exchanges, multiple currencies simultaneously. Such an application can support simulated and real financial trading.

For example, a decision organizer in the Personal Finance Tutor industry can teach users how to buy and sell anything, deal with lenders & creditors, buy insurance, hire contractors, and/or deal with local governments in everyday life.

For example, a decision organizer in the teaching industry can provide simulated situations to teach users “Judgment and Perspective”, that can typically only be gained through years of experience, trial, and error.

For example, a decision organizer in the travel industry can allow users to search across multiple online travel sites of their choosing or a monthly updated profile of sites provided by the decision organizer.

For example, a decision organizer in the academic industry can allow users to choose a course of study and a degree by comparing several colleges, universities, and the careers that follow. Such an embodiment of the present disclosure can include a database of schools and jobs.

For example, a decision organizer in the real estate industry can assist users in learning how to buy or sell a home or any real property. This can include an interactive online database with real life examples and simulated experiences to learn from.

For example, a decision organizer in the management and career industry can allow users to discover what others learn on the corporate fast track from their business mentors in actual decision scenarios. The decision organizer can facilitate interviewing, managing subordinates, and learning how to “manage up”

For example, a decision organizer in the Personal Law industry can allow a user to quickly learn enough to know what a person can and cannot do in simulated everyday situations and when to seek advice from professionals. The decision organizer can provide access to numerous real life and business scenarios.

For example, a decision organizer can have First Aid and Lifesaving application to help a first responder be prepared to help person in need. The present disclosure can provide access to hundreds of scenarios with interactive components and graded video tutorials.

For example, a decision organizer can also be an excellent “interactive” learning tool to show cause and effect simulation. If a user makes the wrong decision, he can get warned of the potential outcome. If the user makes the right decision, he can get rewarded with congratulations. The present disclosure provides interactive learning with harmless consequences through the user of simulated data.

For example, a decision organizer in the medical field can provide a tool for medical professionals to organize the complex choices involved with an end-to-end selection of patient centric services. The decision organizer can be designed to assist in diagnostic, treatment and care planning decisions. The decision organizer can be prepared with guidance from major medical schools and supported by cloud-based Medical Decision Analytics Systems.

In an example of the application of the present disclosure to the medical field, the following scenario can be envisioned. The patient is stabilized and symptoms subside; a fluoroscope shows some potential blockage may be present. A reversible blood thinner and nitroglycerin pills can be prescribed pending an MRI exam and the patient discharged awaiting the results. An example plan might begin at this point for diagnosing: if a coronary blockage is actually present, the decision organizer will provide for using a dye and magnetic resonance imaging. The decision organizer can weigh the variables of cost, availability, or suitability to the patient along with a choice of cardiologist to interpret the results. There are a number of inter dependent choices, some of which are availability, suitability to the specific diagnosis, and economic reasons (e.g. is the provider on the patient's health insurance network).

In some examples, the decision organizer can be pplied to a plurality of specialized care givers, with specific skills and availability, against the requirements of a plurality of organizations, representing patients, requiring those specializations.

For example, a decision organizer in the legal profession can provide for assisting in searching precedent, finding cases that match outcome, or discovering inconsistencies in law across all state boundaries in the US. The decision organizer can include a library of forms and templates and a special heuristic tool for patent law that searches “prior art” and expired patents before 1976.

For example, a decision organizer can provide a product search service, which searches all vendors on the Internet (not just a select few) for items similar to what a receiver requests. The decision organizer can continue to search relentlessly, until the receiver is satisfied. The search continues until the moment the receiver orders. The decision organizer cannot guarantee special offers, availability of products, or stability of vendor prices; rather the decision organizer integrates with the vendors to continually discover such information.

For clarity of explanation, in some instances the present technology can be presented as including individual functional blocks including functional blocks comprising devices, device components, steps or routines in a method embodied in software, or combinations of hardware and software.

In some configurations the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Methods according to the above-described examples can be implemented using computer-executable instructions that are stored or otherwise available from computer readable media. Such instructions can comprise, for example, instructions and data which cause or otherwise configure a general-purpose computer, special purpose computer, or special purpose processing device to perform a certain function or group of functions. Portions of computer resources used can be accessible over a network. The computer executable instructions can be, for example, binaries, intermediate format instructions such as assembly language, firmware, or source code. Examples of computer-readable media that can be used to store instructions, information used, and/or information created during methods according to described examples include magnetic or optical disks, flash memory, USB devices provided with non-volatile memory, networked storage devices, and so on.

Devices implementing methods according to these disclosures can comprise hardware, firmware and/or software, and can take any of a variety of form factors. Typical examples of such form factors include laptops, smart phones, small form factor personal computers, personal digital assistants, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are means for providing the functions described in these disclosures.

While various examples of the present invention have been described above, it should be understood that they have been presented by way of example only, and not limitation. Numerous changes to the disclosed examples can be made in accordance with the disclosure herein without departing from the spirit or scope of the invention. Thus, the breadth and scope of the present invention should not be limited by any of the above described examples. Rather, the scope of the invention should be defined in accordance with the following claims and their equivalents.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The terminology used herein is for the purpose of describing particular examples only and is not intended to be limiting of the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art to which this invention belongs. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

What is claimed is:

1. A system comprising:
an arbitrage system;
a plurality of user terminals associated with a plurality of patients and communicatively coupled to the arbitrage system; and
a plurality of service systems associated with a plurality of medical service providers communicatively coupled to the arbitrage system,
wherein the arbitrage system is configured for:
obtaining, via the user terminals, patient information for each of the plurality of patients, the patient information indicating requirements for medical services and flexibility regarding the requirements;
collecting, from the plurality of service systems, service information for the plurality of medical service providers, the service information comprising available services, costs and availability for the available services, and incentives available, and
for each of the at least one patient, generating one or more proposals for the medical services based on the patient information and the service information, wherein the proposals are generated so as to maximize a utilization of the each of the plurality of medical service providers.

2. A method comprising:
obtaining, via a plurality of user terminals associated with a plurality of patients, patient information for each of the plurality of patients, the patient information indicating requirements for medical services and flexibility regarding the requirements;
collecting, from a plurality of service systems associated with a plurality of medical service providers, service information for the plurality of medical service providers, the service information comprising available services, costs and availability for the available services, and incentives available, and
for each of the at least one patient, generating one or more proposals for the medical services based on the patient information and the service information, wherein the proposals are generated so as to maximize a utilization of the each of the plurality of medical service providers.
